(19) United States
(12) Patent Application Publication
(10) Pub. No.: US 2004/0058957 A1
(43) Pub. Date: Mar. 25, 2004

(54) Benzothiophene derivatives and herbicidal compositions containing the same

(76) Inventors: Seiji Tomita, Chiba (JP); Masatoshi Saitou, Chiba (JP); Hiroki Sekiguchi, Chiba (JP); Shin-ichiro Ogawa, Chiba (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/451,322

(22) PCT Filed: Sep. 7, 2001
(86) PCT No.: PCT/JP01/07779

(30) Foreign Application Priority Data
Dec. 21, 2000 (JP) 2000-388045

(51) Int. Cl. C 07D 4 9/02; C07D 333/52; C07D 333/72; A61K 31/453; A61K 31/4025

(52) U.S. Cl. 514/320; 514/422; 514/443; 546/202; 548/525; 549/51

(57) ABSTRACT

The benzothiophene derivative of the present invention is represented by the general formula (I):

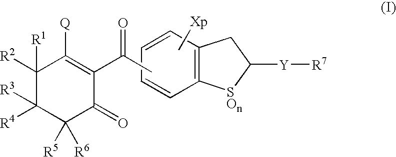

wherein R¹ to R⁷, X, Y, Q, n and p are as defined in the specification. A herbicidal composition containing the benzothiophene derivatives as the effective component is less injury to cultivated crops such as paddy rice and has a broad spectrum of controlling the growth of weeds in a low application rate.

### TECHNICAL FIELD

The present invention relates to novel benzothiophene derivatives and herbicidal compositions containing the benzothiophene derivatives as the herbicidally effective component. More particularly, the present invention relates to benzothiophene derivatives useful as herbicides for controlling undesired cropland weeds and paddy weeds, especially paddy weeds, which are injurious to growth of cultivated plants, and further relates to herbicidal compositions containing the benzothiophene derivatives as the herbicidally effective component.

### BACKGROUND ART

Since herbicides are chemicals important for saving work for weed control and achieving high crop yield, research and development of new herbicides have been extensively conducted for a long time and numerous types of chemicals have been practically used. However, the need still continues for new chemicals having more effective herbicidal activity, particularly, new chemicals which can selectively control only weeds in low application rates without causing injury to field crops and paddy rice.

It is known that certain types of triketone derivatives having a bicyclic benzoyl structure show a high safety to field crops and a good herbicidal activity to cropland weeds. However, among these triketone derivatives having a bicyclic benzoyl structure, a (2,3-dihydrobenzothiophen-5-yl)carbonyl-2,6-cyclohexanedione derivative has not been sufficiently studied, and little is known concerning its herbicidal activity.

Japanese Patent No. 2,579,663 discloses a substituted cyclic dione compound as a herbicidally active compound, and the patented claims include (2,3-dihydrobenzothiophen-5-yl)carbonyl-2,6-cyclohexanedione derivatives. However, the above Japanese Patent fails to describe the details of the derivatives, also fails to practically disclose specific compounds and their herbicidal activity.

The inventors have already found, as (2,3-dihydrobenzothiophen-5-yl)carbonyl-2,6-cyclohexanedione derivatives having a herbicidal activity, the compounds represented by the general formula (II):

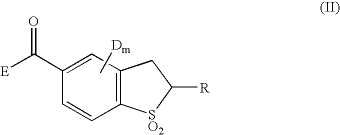

wherein E represents a 1,3-cyclohexanedione derivative-2-yl group; D represents a substituent; R represents hydrogen or lower alkyl; and m is an integer of 0 to 2 (WO 00/20408). Although the above compounds represented by the general formula (II) exhibit a low injury to cultivated crops, especially paddy rice, and are capable of controlling various weeds in low application rates, there is a further need to develop new compounds showing a still lower injury to cultivated crops such as paddy rice and having a broad spectrum of controlling weeds even in low application rates.

### DISCLOSURE OF INVENTION

An object of the present invention is to provide novel compounds exhibiting an extremely low injury to cultivated crops, especially paddy rice, and having a broad spectrum of controlling various weeds in low application rates, and to provide herbicidal compositions comprising the compounds as a herbicidally effective component, especially herbicidal compositions useful for controlling paddy weeds.

As the result of extensive study, the inventors have found that the above object is achieved by (2,3-dihydrobenzothiophen-5-yl)carbonyl-2,6-cyclohexane-dione derivatives having an alkyl substituent group bonded to the 2-position of its benzothiophene ring via O, S, SO or SO₂. The present invention has been accomplished based on this finding.

Thus, the present invention provides a benzothiophene derivative represented by the following general formula (I):

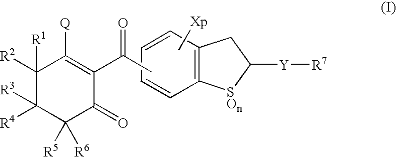

wherein:

each of R¹ to R⁶ independently represents hydrogen, halogen, C₁-C₆ alkyl or C₁-C₆ haloalkyl, and any two of R¹ to R⁶ may be bonded to each other to form a bicyclic ring structure together with a cyclohexene ring;

R⁷ represents C₁-C₆ alkyl which may be substituted;

Q represents hydroxyl or halogen; or Q represents C₁-C₆ alkoxyl, C₁-C₆ alkylthio, C₁-C₆ alkylsulfinyl, C₁-C₆ alkylsulfonyl, phenoxy, phenylthio, phenylsulfinyl, phenylsulfonyl, C₂-C₁₂ dialkylamino, C₂-C₁₂ N-alkoxyalkylamino, five- or six-membered nitrogen-containing heterocyclic residue which can be bonded through its nitrogen, five- or six-membered heterocyclic sulfide group, malonic ester residue which can be bonded at its α-position, β-ketoester residue which can be bonded at its α-position, or β-diketone residue which can be bonded at its α-position, each optionally being substituted;

X represents halogen, nitro, cyano, R⁸, OR⁸, SR⁸, SO₂R⁸ or NR⁸R⁹;

each of R⁸ and R⁹ represents hydrogen; or each of R⁸ and R⁹ represents C₁-C₆ alkyl, C₂-C₆ alkenyl, C₂-C₆ alkynyl, phenyl or benzyl, each being optionally substituted, and R⁸ and R⁹ of NR⁸R⁹ may be the same or different and may be bonded to each other to form a ring structure;

Y represents O, S, SO or SO₂;

n represents 0, 1 or 2; and

p represents 1 or 2.

The present invention further provides a herbicidal composition comprising the benzothiophene derivative as a herbicidally effective ingredient.

### BEST MODE FOR CARRYING OUT THE INVENTION

The benzothiophene derivative of the present invention is represented by the general formula (I):

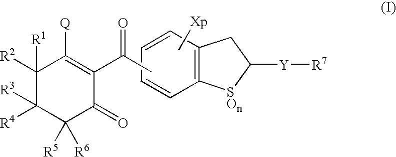

In the general formula (I), each of R¹ to R⁶ independently represents hydrogen, halogen, C₁-C₆ alkyl or C₁-C₆ haloalkyl. Examples of the halogen include fluorine, chlorine, bromine and iodine. Examples of the C₁-C₆ alkyl are methyl, ethyl, and various propyl, butyl, pentyl and hexyl. The various propyl, butyl, pentyl and hexyl may include straight-chain, branched and cyclic isomers. Examples of the C₁-C₆ haloalkyl include those obtained by replacing a part or whole of hydrogen atoms of the above C₁-C₆ alkyl with halogen such as fluorine, bromine and iodine. Specific examples of the C₁-C₆ haloalkyl include chloromethyl, difluoromethyl, trichloromethyl, trifluoromethyl, 2-chloroethyl, 2-fluoroethyl, 3-chloropropyl and 3-fluoropropyl. Of these, preferred R¹ to R⁶ are hydrogen atoms. Any two of R¹ to R⁶ may be bonded to each other to form a bicyclic ring such as bicyclo[3.2.1]octane together with the cyclohexene ring.

R⁷ represents substituted or unsubstituted C₁-C₆ alkyl. Examples of the substituted or unsubstituted C₁-C₆ alkyl include the C₁-C₆ alkyl or substituted C₁-C₆ alkyl for R¹ to R⁶ mentioned above. Specific examples thereof include methyl, ethyl, isopropyl, propyl, 2-methoxyethyl, 2,2-dimethoxyethyl, 2-oxoethyl, 2-methoxyiminoethyl, 2-hydroxyiminoethyl and methoxycarbonylmethyl, with methyl, ethyl, 2-methoxyethyl, 2-methoxyiminoethyl and methoxycarbonylmethyl being preferred.

Q represents hydroxyl or halogen; or Q represents C₁-C₆ alkoxyl, C₁-C₆ alkylthio, C₁-C₆ alkylsulfinyl, C₁-C₆ alkylsulfonyl, phenoxy, phenylthio, phenylsulfinyl, phenylsulfonyl, C₂-C₁₂ dialkylamino, C₂-C₁₂ N-alkoxyalkylamino, five- or six-membered nitrogen-containing heterocyclic residue which can be bonded through its nitrogen, five- or six-membered heterocyclic sulfide group, malonic acid ester residue which can be bonded at its α-position, β-ketoester residue which can be bonded at its α-position, or β-diketone residue which can be bonded at its α-position, each optionally being substituted. Examples of the halogen include fluorine, chlorine, bromine and iodine. Examples of the substituted or unsubstituted C₁-C₆ alkoxyl include methoxy, ethoxy, isopropoxy, 2-chloroethoxy and 2-methoxyethoxy. Examples of the substituted or unsubstituted C₁-C₆ alkylthio include methylthio, ethylthio, 2-hydroxyethylthio, 2-cyanoethylthio, 2-hydroxypropylthio, 2-acetoxyethylthio, 2-methanesulfonylethylthio and 2-acetylaminoethylthio. Examples of the substituted or unsubstituted C₁-C₆ alkylsulfinyl include methylsulfinyl, ethylsulfinyl, 2-hydroxyethylsulfinyl, 2-cyanoethylsulfinyl, 2-hydroxypropylsulfinyl, 2-acetoxyethylsulfinyl, 2-methanesulfonylethylsulfinyl and 2-acetylaminoethylsulfinyl. Examples of the substituted or unsubstituted C₁-C₆ alkylsulfonyl include methylsulfonyl, ethylsulfonyl, 2-hydroxyethylsulfonyl, 2-cyanoethylsulfonyl, 2-hydroxypropylsulfonyl, 2-acetoxyethylsulfonyl, 2-methanesulfonylethylsulfonyl and 2-acetylaminoethylsulfonyl. Examples of the substituted or unsubstituted phenoxy include phenoxy, 4-methylphenoxy and 4-hydroxyphenoxy. Examples of the substituted or unsubstituted phenylthio include phenylthio, 4-methylphenylthio and 4-hydroxyphenylthio. Examples of the substituted or unsubstituted phenylsulfinyl include phenylsulfinyl, 4-methylphenylsulfinyl and 4-hydroxyphenylsulfinyl. Examples of the substituted or unsubstituted phenylsulfonyl include phenylsulfonyl, 4-methylphenylsulfonyl and 4-hydroxyphenylsulfonyl.

Examples of the substituted or unsubstituted C₂-C₁₂ dialkylamino include dimethylamino, diethylamino, diisopropylamino and dicyclohexylamino. Examples of the substituted or unsubstituted C₂-C₁₂ N-alkoxyalkylamino include N-methoxymethylamino. Examples of the substituted or unsubstituted five- or six-membered nitrogen-containing heterocyclic residue which can be bonded through its nitrogen include residues of nitrogen-containing heterocyclic compounds such as pyrrolidine, piperidine, morpholine, pyrazole, 3-methylpyrazole, 1,2,4-triazole and 4,5-dihydropyrazole. Examples of the substituted or unsubstituted five- or six-membered heterocyclic sulfide group include 2-pyridinylthio, 2-pyrimidinylthio, (4-methylpyrimidin-2-yl)thio, 2-thiophenylthio, (1,3-thiazol-2-yl)thio, (2-methylfuran-3-yl)thio and (1-methylimidazol-2-yl)thio. Examples of the malonic acid ester residue which can be bonded at its α-position include dimethyl malonate residue and diethyl malonate residue. Examples of the β-ketoester residue which can be bonded at its α-position include methyl acetoacetate residue and ethyl acetoacetate residue. Examples of the β-diketone residue which can be bonded at its α-position include acetylacetone residue and 1,3-cyclohexanedione residue. Of these, preferred Q is hydroxyl.

X represents halogen, nitro, cyano, R⁸, OR⁸, SR⁸, SO₂R⁸ or NR⁸R⁹. Each of R⁸ and R⁹ independently represents hydrogen, or each of R⁸ and R⁹ independently represents C₁-C₆ alkyl, C₂-C₆ alkenyl, C₂-C₆ alkynyl, phenyl or benzyl, each being optionally substituted. Examples of the substituted or unsubstituted C₁-C₆ alkyl include methyl, ethyl, isopropyl, cyclopropyl, cyclohexyl, trichloromethyl, difluoromethyl, trifluoromethyl and methoxymethyl. Examples of the substituted or unsubstituted C₂-C₆ alkenyl include ethenyl, allyl, 1-propenyl and 1-ethoxyethenyl. Examples of the substituted or unsubstituted C₂-C₆ alkynyl include ethynyl, 1-propynyl and 2-propynyl. Examples of the substituted or unsubstituted phenyl include phenyl and 4-methylphenyl. Examples of the substituted or unsubstituted benzyl include benzyl and α-methylbenzyl. R⁸ and R⁹ of NR⁸R⁹ may be the same or different and may be bonded to each other to form a ring such as pyrrolidine ring, piperidine ring and morpholine ring. Of these substituents for X, preferred are chlorine, bromine, nitro, cyano and substituted or unsubstituted C₁-C₆ alkyl, and more preferred are methyl, ethyl, trifluoromethyl and methoxymethyl.

Y represents O, S, SO or SO₂, preferably S, SO or SO₂; subscript n represents 0, 1 or 2, preferably 2; and subscript p represents 1 or 2.

Of the benzothiophene derivatives represented by the general formula (I), especially preferred are those represented by the following general formula (I-a):

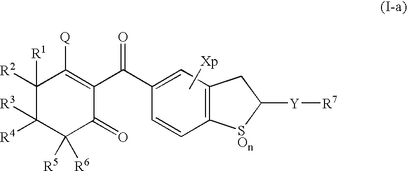

wherein R¹ to R⁷, Q, X, Y, n and p are the same as defined above.

The benzothiophene derivatives represented by the general formula (I) wherein Q is hydroxyl have the following tautomers. The benzothiophene derivatives of the present invention include these tautomers and mixtures thereof.

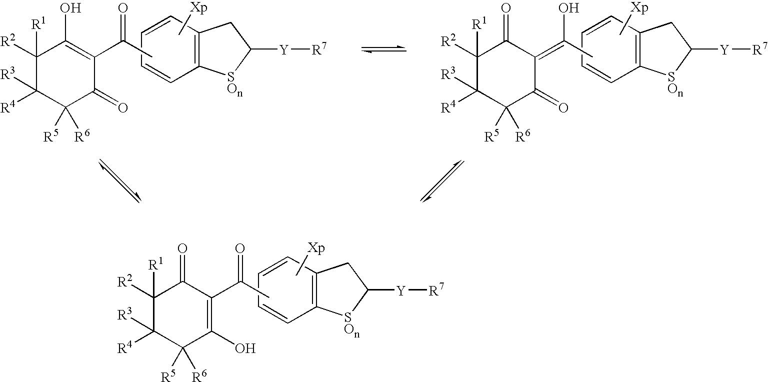

The benzothiophene derivatives represented by the general formula (I) wherein R¹ or R⁶ is hydrogen have the following tautomers. The benzothiophene derivatives of the present invention include these tautomers and mixtures thereof.

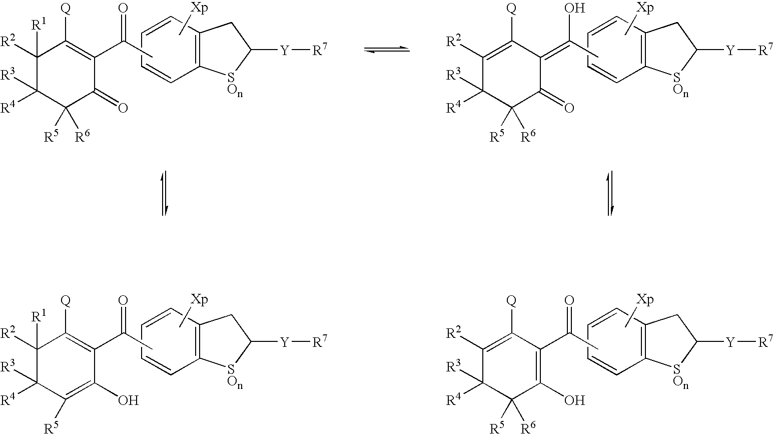

The benzothiophene derivatives represented by the above general formula (I) according to the present invention may be produced by the following method described in WO 00/20408 except for the introduction of a substituent into the 2-position of the benzothiophene.

(1) A carboxylic acid compound (III) produced by the method described in WO 00/20408 is reacted with an alcohol to obtain an ester compound (IV).

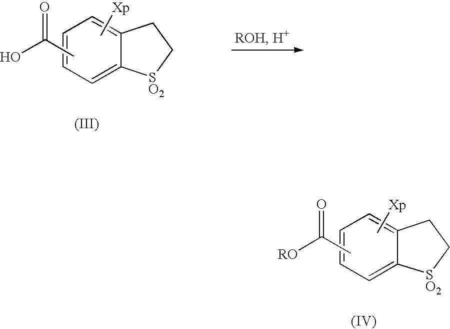

wherein ROH is lower alcohol such as methanol and ethanol; and X and p are the same as defined above.

The above reaction is performed in the presence of a catalytic amount of a protonic acid such as sulfuric acid and p-toluenesulfonic acid. As the alcohol, methanol or ethanol may be suitably used in an equimolar amount or higher. Also, the above reaction may be performed in the presence or absence of a solvent. As the solvent, there may be used inert solvents such as benzene and toluene. The reaction is carried out at a temperature between room temperature and a boiling point of the solvent by continuously stirring until the reaction is completed, while removing water produced during the reaction.

(2) The ester compound (IV) produced in the step (1) is reacted with a halogenating agent to obtain a halogenated compound (V).

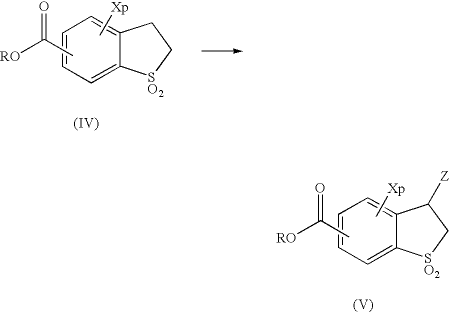

wherein Z represents a halogen atom.

The above reaction is performed in the presence of an inert solvent such as carbon tetrachloride using a halogenating agent such as N-bromosuccinimide (NBS) in an equimolar amount or higher. As the radical source, a catalytic amount of azobisisobutylonitrile (AIBN), etc. is used. The reaction is carried at a boiling point of the solvent by stirring until the reaction is completed.

(3) The halogenated compound (V) obtained in the step (2) is subjected to dehydrohalogenation to obtain a benzothiophene compound (VI).

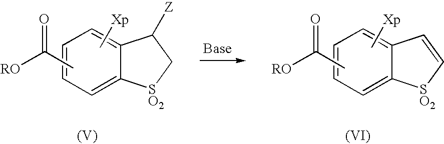

The above reaction is performed in the presence of an inert solvent such as methylene chloride using a base such as triethylamine in an equimolar amount or higher. The reaction is carried out at a temperature of from 0° C. to a boiling point of the solvent by stirring until the reaction is completed.

(4) A substituent is introduced into the 2-position of the benzothiophene compound (VI) obtained in the step (3) to obtain a compound (VII).

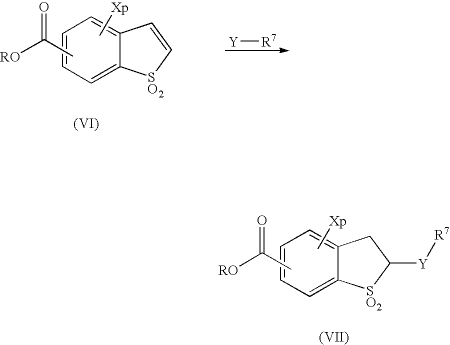

wherein R⁷ and Y are the same as defined above.

The above reaction may be performed in a two-phase system composed of water and an inert solvent such as methylene chloride using a nucleophilic agent such as sodium salt of methane thiol in an equimolar amount or higher. A catalytic amount of a phase transfer catalyst such as tetrabutylammonium bromide (TBAB) is also used. The reaction is carried out at a temperature between room temperature and a boiling point of the solvent by stirring until the reaction is completed.

(5) The compound (VII) obtained is the step (4) is hydrolyzed to a carboxylic acid compound (VIII).

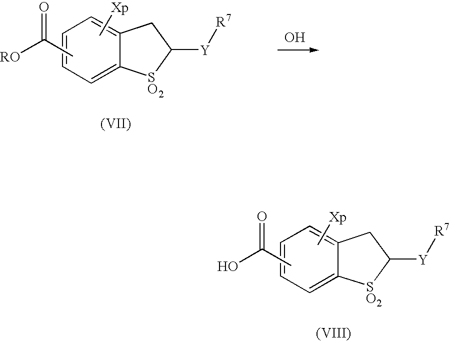

The above reaction is performed in a mixed protonic solvent such as water and methanol or ethanol with or without adding an inert solvent such as methylene chloride, using a base such as sodium hydroxide and lithium hydroxide in an equimolar amount or higher. The reaction is carried out at a temperature of from 0° C. to a boiling point of the solvent by stirring until the reaction is completed.

The benzothiophene derivative represented by the general formula (I) according to the present invention is produced from the carboxylic acid compound (VIII), for example, by the method described in WO00/20408.

The herbicidal composition of the present invention contains the resultant benzothiophene derivatives represented by the genera formula (I) as the herbicidally effective component, and is especially suitable for use in paddy field. The benzothiophene derivatives may be formulated into wettable powders, emulsifiable concentrates, dusts and granules by mixing with a liquid carrier such as a solvent or a solid carrier such as a fine mineral powder. A surfactant may be added to improve the emulsifying property, the dispersing property and the spreading property of the herbicidal composition.

Wettable powders can be prepared, in general, by blending 5 to 55% by weight of the benzothiophene derivative, 40 to 93% by weight of a solid carrier and 2 to 5% by weight of a surfactant. Emulsifiable concentrates can be prepared, in general, by mixing 10 to 50% by weight of the benzothiophene derivative, 35 to 85% by weight of a solvent and 5 to 15% by weight of a surfactant. Dusts can be prepared, in general, by blending 1 to 15% by weight of the benzothiophene derivative, 80 to 97% by weight of a solid carrier and 2 to 5% by weight of a surfactant. Granules can be prepared, in general, by blending 1 to 15% by weight of the benzothiophene derivative, 80 to 97% by weight of a solid carrier and 2 to 5% by weight of a surfactant and granulating the resultant mixture.

The solid carrier is preferably a fine powder of minerals such as oxides (diatomaceous earth and slaked lime), phosphates (apatite), sulfates (gypsum), talc, pyroferrite, clay, kaolin, bentonite, acidic white clay, white carbon, quartz and silica.

As the solvent, there may be used organic solvents including aromatic hydrocarbons such as benzene, toluene and xylene; chlorinated hydrocarbons such as o-chlorotoluene, trichloroethane and trichloroethylene; alcohols such as cyclohexanol, amyl alcohol and ethylene glycol; ketones such as isophorone, cyclohexanone and cyclohexenylcyclohexanone; ethers such as butylcellosolve, diethyl ether and methyl ethyl ether; esters such as isopropyl acetate, benzyl acetate and methyl phthalate; amides such as dimethylformamide; and mixtures thereof.

As the surfactant, usable are any of anionic surfactants, nonionic surfactants, cationic surfactants, and amphoteric surfactants such as amino acids and betaine.

In addition to the benzothiophene derivatives, the herbicidal compositions of the present invention may contain, if necessary, another herbicidally active component selected from a diphenyl ether herbicide, a triazine herbicide, an urea herbicide, a carbamate herbicide, a thiocarbamate herbicide, an acid anilide herbicide, a pyrazole herbicide, a phosphoric acid herbicide, a sulfonylurea herbicide and an oxadiazone herbicide. The herbicidal compositions of the present invention may further contain, if necessary, an insecticide, an antibiotic, a plant growth regulator and a fertilizer.

The herbicidal compositions of the present invention are applied to weeds before or after germination or their environments. The modes of application vary depending on the type of the cultivated plants and the environmental factors. For example, the herbicidal compositions may be applied by spraying, scattering, sprinkling or irrigating.

An application rate of the azole compounds of the present invention is determined by a number of factors such as formulation selected, mode of application, amount and type of weed species, growing conditions, etc. In general, the application rate is 0.025 to 5 kg/ha, preferably 0.05 to 2 kg/ha. One skilled in the art can easily determine the application rate necessary for the desired level of weed control.

The herbicidal compositions of the present invention are effective for controlling weeds in useful cultivated plants such as Gramineous crops such as rice, wheat, barley, corn, oat and sorghum; broad-leaved crops such as soy bean, cotton, beet, sunflower and rapeseed; fruit trees; vegetables such as fruit vegetables, root vegetables and leaf vegetables; and turf.

The herbicidal composition of the present invention are effective for controlling paddy weeds, e.g., Alismataceous weeds such as Alisina canaliculatum, Sagittaria tifolia and Sagittaria pygmaea; Cyperaceous weeds such as Cyperus difformis, Cyperus serotinus, Scirpusjuncoides and Eleocharis kuroguwai; Scrophulariaceous weeds such as Lindernia pyxidaria; Pontenderiaceous weeds such as Monochoria vaginalis; Potamogetonaceous weeds such as Potamogeton distinctus; Lythraceous weeds such as Rotala indica; and Gramineous weeds such as Echinochloa crus-galli.

Cropland weeds include broad-leaved weeds and narrow-leaved weeds. Broad-leaved weeds may be Solanaceous weeds such as Solanum nigrum and Datura stramoniuum; Malvaceous weeds such as Abutilon theophrasti and Sida spinosa; Convolvulaceous weeds such as Ipomoea purpurea; Amaranthaceous weeds such as Amaranthus lividus; Composite weeds such as Xanthium strumarium, Amhrosia artemisifolia, Galinsoga ciliata, Cirsium arvense, Senecio vulgaris and Erigeron annus; Brasicaceous weeds such as Rorippa indica, Sinapis arvensis and Capsella hursa-pastoris; Polygonaceous weeds such as Polygonum hulumei and Polygonum convolvulus; Portulacaceous weeds such as Portulaca oleracea; Chenopodiaceous weeds such as Chenopodium aluhum, Chenopodiumr ficiolium and Kochia scoparia; Caryophyllaceous weeds such as Stellaria media; Scrophulariaceous weeds such as Veronica persica; Commelinaceous weeds such as Commelina communis; Euphorbiaceous weeds such as Lamium amplexicaule, Euphorhia supina and Euphorhia maculata; Rubiaceous weeds such as Galium spurium, Galium aparine and Rubia akane; Violaceous weeds such as Viola arvensis; and Leguminous weeds such as Sesbania exaltata and Cassia obtusifolia. Narrow-leaved weeds may be Graminaceous weeds such as Sorghum bicolor, Panicum dichotomifloruin, Sorghtum haepense, Echinochloa crus-galli, Digitaria adscendens, Avena fatua, Eleusine indica, Setaria viridis and Alopecurus aequalis; and Cyperaceous weeds such as Cyperus rotundus and Cypeus esculentus.

The present invention will be described more specifically with reference to the following preparation examples and weed control examples. However, these examples are not intended to limit the present invention thereto.

### EXAMPLE 1

[1] Preparation of 4-chloro-5-oxycarbonyl-2-methylthio-2,3-dihydrobenzothiophene 1,1-dioxide

(1) Preparation of 4-chloro-5-ethoxycarbonyl-2,3-dihydrobenzothiophene 1,1-dioxide

After dissolving 3.0 g of 4-chloro-5-oxycarbonyl-2,3-dihydrobenzothiophene 1,1-dioxide in 15 ml of ethanol, 0.1 g of p-toluenesulfonic acid was added. The mixture was refluxed under heating for 12 h using a reflux tube packed with molecular sieve 4A. The reaction solution was cooled to room temperature, and then mixed with water to precipitate solids. The precipitated solids were filtered and then dried to obtain 3.2 g of the target ester compound.

(2) Preparation of 4-chloro-5-ethoxycarbonyl-3-bromo-2,3-dihydrobenzothiophene 1,1-dioxide

After suspending 1.0 g of the above ester compound and 0.71 g of NBS in 10 ml of carbon tetrachloride, 0.1 g of AIBN was added. The mixture was refluxed under heating for 16 hours. The resultant reaction solution was mixed with water, and then extracted with ethyl acetate. The extract was washed with an aqueous sodium carbonate solution and saturated brine, and then dried over anhydrous sodium sulfate. Then, the solvent was distilled away under reduced pressure to obtain 1.3 g of the target brominated compound.

(3) Preparation of 4-chloro-5-ethoxycarbonylbenzothiophene 1,1-dioxide

A solution of 1 ml of triethylamine in 5 ml of methylene chloride was added dropwise at room temperature into a solution of 2.1 g of the above brominated compound in 15 ml of methylene chloride. The resultant mixture was stirred at room temperature for 1.5 h. Then, the mixture was added with 5% hydrochloric acid and extracted with methylene chloride. The extract was dried over anhydrous sodium sulfate. Then, the solvent was distilled away under reduced pressure to obtain 1.7 g of the target benzothiophene compound.

(4) Preparation of 4-chloro-5-ethoxycarbonyl-2-methylthio-2,3-dihydrobenzothiophene 1,1-dioxide

After dissolving 4.0 g of the above benzothiophene compound and TBAB in 30 ml of methylene chloride, 10 ml of a 15% aqueous solution of sodium salt of methanethiol was added at room temperature, followed by stirring for 4 h. The resultant reaction solution was added with water, and then extracted with methylene chloride. The extract was dried over anhydrous sodium sulfate. Then, the solvent was distilled away under reduced pressure to obtain a crude product, which was then purified by column chromatography to obtain 2.3 g of the target sulfide compound.

(5) Preparation of 4-chloro -5-oxycarbonyl-2-methylthio-2,3-dihydrobenzothiophene-1,1-dioxide

A solution of 2.5 g of the above sulfide compound in a mixed solvent of 12.4 ml of methylene chloride, 12.4 ml of methanol and 6.2 ml of water was added at room temperature with 0.39 g of lithium hydroxide monohydrate, followed by stirring for 4 h. The resultant reaction solution was added with 5% hydrochloric acid and then extracted with ethyl acetate. The extract was dried over anhydrous sodium sulfate. Then, the solvent was distilled away under reduced pressure to obtain 2.2 g of the target carboxylic acid compound.

¹H-NMR [ppm; acetone-d⁶ (TMS standard)] of the carboxylic acid compound showed the following absorption peaks: 2.43 (3H, s), 3.17 (1H, dd), 3.87 (1H, dd), 4.79 (1H, dd), 7.83 (1H, d) and 8.05 (1H, d). As a result, it was confirmed that the obtained compound was 4-chloro-5-oxycarbonyl-2-methylthio-2,3-dihydrobenzothiophene 1,1-dioxide.

[2] Preparation of 4-chloro-2-methylthio-5-(1,3-dioxocyclohex-2-yl)carbonyl-2,3-dihydrobenzothiophene 1,1-dioxide (Compound No. 1)

After suspending 1.8 g of 4-chloro-5-oxycarbonyl-2-methylthio-2,3-dihydrobenzothiophene 1,1-dioxide in 10 ml of 1,2-dichloroethane, 0.55 ml of thionyl chloride and 0.05 ml of N,N-dimethylformamide (DMF) were added. The mixture was refluxed under heating for one hour. Then, the reaction solution was distilled under reduced pressure to remove the solvent therefrom. The distillation residue was mixed with a solution of 0.85 g of 1,3-cyclohexanedione in 10 ml of acetonitrile, and then added dropwise with a solution of 2.2 ml of triethylamine in 5 ml of acetonitrile at room temperature. The mixture was stirred at room temperature for 2 h, and then mixed with 0.1 ml of acetone cyanohydrin and further stirred at room temperature for 7 h. The reaction solution was mixed with a 10% sodium hydroxide aqueous solution, and then washed with methylene chloride. After making the aqueous phase acidic by concentrated sulfuric acid, the aqueous phase was extracted with methylene chloride. The extract was dried over anhydrous sodium sulfate, and then, the solvent was distilled away under reduced pressure to obtain 1.6 g of the target compound.

¹H-NMR data and IR spectra data of the compound obtained at the above step [2] together with the results of measurements of chemical structure and melting point are shown in Table 1. As a result of these measurements, it was confirmed that the obtained compound was 4-chloro-2-methylthio-5-(1,3-dioxocyclohex-2-yl)carbonyl-2,3-dihydrobenzothiophene 1,1-dioxide (Compound No. 1).

### EXAMPLE 2

[1] Preparation of 4-chloro-2-ethylthio-5-(1,3-cdioxocyclohex-2-yl)carbonyl-2,3-cdihydrobenzothiophene 1,1-dioxide (Compound No. 2)

The same procedure as in Example 1-[2] was repeated except that 4-chloro-5-oxycarbonyl-2-ethylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 3

[1] Preparation of 4-chloro-2-(2-propyl)thio-5-(1,3-dioxocyclohex-2-yl)carbonyl-2,3-dihydrobenzothiophene 1,1-dioxide (Compound No. 3)

The same procedure as in Example 1-[2] was repeated except that 4-chloro-5-oxycarbonyl-2-(2-propyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 4

[1] Preparation of 4-chloro-2-(1-propyl)thio-5-(1,3-dioxocyclohex-2-yl)carbonyl-2,3-dihydrobenzothiophene 1,1-dioxide (Compound No. 4)

The same procedure as in Example 1-[2] was repeated except that 4-chloro-5-oxycarbonyl-2-(1-propyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 5

[1] Preparation of 4-chloro-2-(2-methoxyethyl)thio-5-(1,3-dioxocyclohex-2-yl)carbonyl-2,3-dihydrobenzothiophene 1,1-dioxide (Compound No. 5)

The same procedure as in Example 1-[2] was repeated except that 4-chloro-5-oxycarbonyl-2-(2-methoxyethyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 6

[1] Preparation of 4-chloro-2-(2,2-diethoxyethyl)thio-5-(1,3-dioxocyclohex-2-yl)carbonyl-2,3-dihydrobenzothiophene 1,1-dioxide (Compound No. 6)

The same procedure as in Example 1-[2] was repeated except that 4-chloro-5-oxycarbonyl-2-(2,2-diethoxyethyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 7

[1] Preparation of 4-chloro-2-(2-oxoethyl)thio-5-(1,3-dioxocyclohex-2-yl)carbonyl-2,3-dihydrobenzothiophene 1,1-dioxide (Compound No. 7)

The same procedure as in Example 1-[2] was repeated except that 4-chloro-5-oxycarbonyl-2-(2-oxoethyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 8

[1] Preparation of 4-chloro-2-(2-methoxyiminoethyl)thio-5-(1,3-dioxocyclohex-2-yl)carbonyl-2,3-dihydrobenzothiophene 1,1-dioxide (Compound No. 8)

The same procedure as in Example 1-[2] was repeated except that 4-chloro-5-oxycarbonyl-2-(2-methoxyiminoethyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 9

[1] Preparation of 4-chloro-2-(2-hydroxyiminoethyl)thio-5-(1,3-dioxocyclohex-2-yl)carbonyl-2,3-dihydrobenzothiophene 1,1-dioxide (Compound No. 9)

The same procedure as in Example 1-[2] was repeated except that 4-chloro-5-oxycarbonyl-2-(2-hydroxyiminoethyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 10

[1] Preparation of 4-methyl-2-methylthio-5-(1,3-dioxocyclohex-2-yl)carbonyl-2,3-dihydrobenzothiophene 1,1-dioxide (Compound No. 10)

The same procedure as in Example 1-[2] was repeated except that 4-methyl-5-oxycarbonyl-2-methylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 11

[1] Preparation of 2-methylthio-4-trifluoromethyl-5-(1,3-dioxocyclohex-2-yl)carbonyl-2,3-dihydrobenzothiophene 1,1-dioxide (Compound No. 11)

The same procedure as in Example 1-[2] was repeated except that 5-oxycarbonyl-2-methylthio-4-trifluoromethyl-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 12

[1] Preparation of 4-methoxy-2-methylthio-5-(1,3-dioxocyclohex-2-yl)carbonyl-2,3-dihydrobenzothiophene 1,1-dioxide (Compound No. 12)

The same procedure as in Example 1-[2] was repeated except that 4-methoxy-5-oxycarbonyl-2-methylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 13

[1] Preparation of 4-chloro-2-methoxy-5-(1,3-dioxocyclohex-2-yl)carbonyl-2,3-dihydrobenzothiophene 1,1-dioxide (Compound No. 13)

The same procedure as in Example 1-[2] was repeated except that 4-chloro-2-methoxy-5-oxycarbonyl-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 14

[1] Preparation of 4-chloro-2-methoxy-5-(4,4-dimethyl-1,3-dioxocyclohex-2-yl)carbonyl-2,3-dihydro benzothiophene 1,1-dioxide (Compound No. 14)

The same procedure as in Example 1-[2] was repeated except that 4,4-dimethyl-1,3-cyclohexanedione was used in place of 1,3-cyclohexanedione, thereby obtaining the target compound. ¹H-NMR data and IR spectra data of the obtained compound together with the results of measurements of chemical structure and melting point are shown in Table 1.

### EXAMPLE 15

[1] Preparation of 4-chloro-2-methanesulfonyl-5-(1,3-dioxocyclohex-2-yl)carbonyl-2,3-dihydrobenzothiophene 1,1-dioxide (Compound No. 15)

One gram of 4-chloro-2-methylthio-5-(1,3-dioxocyclohex-2-yl)carbonyl-2,3-dihydrobenzothiophene 1,1-dioxide produced in Example 1-[2] was dissolved in 5 ml of acetic acid. The obtained solution was added with 0.6 ml of a 30% hydrogen peroxide aqueous solution and stirred at 60° C. for 2 h. The resultant reaction solution was added with water to precipitate solids, which were filtered and then dried to obtain 1.0 g of the target compound. ¹H-NMR data and IR spectra data of the obtained compound together with the results of measurements of chemical structure and melting point are shown in Table 1.

### EXAMPLE 16

[1] Preparation of 4-chloro-2-(1-propane)sulfonyl-5-(1,3-dioxocyclohex-2-ylcarbony1-2,3-dihycrobenzothiophene 1,1-dioxide (Compound No. 16)

The same procedure as in Example 15-[1] was repeated except that 4-chloro-2-(1-propyl)thio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 17

[1] Preparation of 4-chloro-2-methylthio-5-(3-chloro-1-oxo-2-cyclohexen-2-yl)carbonyl-2,3-dihydrobenzothiophene 1,1-dioxide (Compound No. 17)

After dissolving 1.0 g of 4-chloro-2-methylthio-5-(1,3-dioxocyclohex-2-yl)carbonyl-2,3-dihydrobenzothiophene 1,1-dioxide produced in Example 1-[2] in 5 ml of 1,2-dichloroethane, 0.36 g of oxalyl chloride and 0.05 ml of DMF were added. The mixture was stirred at 60° C. for one hour. Then, the solvent was distilled away under reduced pressure, and the obtained crude product was purified by column chromatography to obtain 1.0 g of the target compound. ¹H-NMR data and IR spectra data of the obtained compound together with the results of measurements of chemical structure and melting point are shown in Table 1.

### EXAMPLE 18

[1] Preparation of 4-chloro-2-methanesulfonyl-5-(3-chloro-1-oxo-2-cyclohexen-2-yl)carbonyl-2,3-dihydrobenzothiophene 1,1-dioxide (Compound No. 18)

The same procedure as in Example 17-[1] was repeated except that 4-chloro-2-methanesulfonyl-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 19

[1] Preparation of 4-chloro-2-ethylthio-5-(3-chloro-1-oxo-2-cyclohexen-2-yl)carbonyl-2,3-dihydrobenzothiophene 1,1-dioxide (Compound No. 19)

The same procedure as in Example 17-[1] was repeated except that 4-chloro-2-ethylthio-5-(1,3-dioxocyclohex-2-yl) carbonyl-2,3-dihydrobenzothiophene 1,1-dioxide was used in place of 4-chloro-2-methylthio-5-(1,3-cdioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 20

[1] Preparation of 4-chloro-2-ethanesulfonyl-5-(3-chloro-1-oxo-2-cyclohexen-2-yl)carbonyl-2,3-dihydrobenzothiophene 1,1-dioxide (Compound No. 20)

The same procedure as in Example 17-[1] was repeated except that 4-chloro-2-ethanesulfonyl-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 21

[1] Preparation of 4-chloro-2-(2-propyl)thio-5-(3-chloro-1-oxo-2-cyclohexen-2-yl)carbonyl-2,3-dihydrobenzothiophene 1,1-dioxide (Compound No. 21)

The same procedure as in Example 17-[1] was repeated except that 4-chloro-2-(2-propyl)thio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 22

[1] Preparation of 4-chloro-2-(2-propane)sulfonyl-5-(3-chloro-1-oxo-2-cyclohexen-2-yl)carbonyl-2,3-dihydrobenzothiophene 1,1-dioxide (Compound No. 22)

The same procedure as in Example 17-[1] was repeated except that 4-chloro-2-(2-propane)sulfonyl-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 23

[1] Preparation of 4-chloro-2-(1-propyl)thio-5-(3-chloro-1-oxo-2-cyclohexen-2-yl)carbonyl-2,3-dihrobenzothiophene 1,1-dioxide (Compound No. 23)

The same procedure as in Example 17-[1] was repeated except that 4-chloro-2-(2-propyl)thio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 24

[1] Preparation of 4-chloro-2-(1-propane)sulfonyl-5-(3-chloro-1-oxo-2-cyclohexen-2-yl)carbonyl-2,3-dihydrobenzothiophene 1,1-dioxide (Compound No. 24)

The same procedure as in Example 17-[1] was repeated except that 4-chloro-2-(1-propane)sulfonyl-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 25

[1] Preparation of 4-chloro-2-(2-methoxyethyl)thio-5-(3-chloro-1-oxo-2-cyclohexen-2-yl)carbonyl-2, 3-dihydrobenzothiophene 1,1-dioxide (Compound No. 25)

The same procedure as in Example 17-[1] was repeated except that 4-chloro-2-(2-methoxyethyl)thio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 26

[1] Preparation of 4-chloro-2-(2-methoxyethane)sulfonyl-5-(3-chloro-1-oxo-2-cyclohexen-2-yl)carbonyl-2,3-dihydrobenzothiophene 1,1-dioxide (Compound No. 26)

The same procedure as in Example 17-[1] was repeated except that 4-chloro-2-(2-methoxyethane)sulfonyl-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 27

[1] Preparation of 4-chloro-2-(methoxycarbonylmethyl)thio-5-(3-chloro-1-oxo-2-cyclohexen-2-yl)carbonyl-2,3-dihydrobenzothiophene 1,1-dioxide (Compound No. 27)

The same procedure as in Example 17-[1] was repeated except that 4-chloro-2-(methoxycarbonylmethyl)thio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 28

[1] Preparation of 4-chloro-2-methanesulfinyl-5-(3-chloro-1-oxo-2-cyclohexen-2-yl)carbonyl-2,3-dihydrobenzothiophene 1,1-dioxide (Compound No. 28)

The same procedure as in Example 17-[1] was repeated except that 4-chloro-2-methanesulfinyl-5-(1,3-dioxocyclohex-2-yl)carbonyl-2,3-dihydrobenzothiophene 1,1-dioxide was used in place of 4-chloro-2-methylthio-5-(1,3-cdioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 29

[1] Preparation of 4-chloro-2-methylthio-5-[3-(4-methylphenyl)thio-1-oxo-2-cyclohexen-2-yl]carbonyl-2,3-dihydrobenzothiophene 1,1-dioxide (Compound No. 29)

After dissolving 0.50 g of 4-chloro-2-methylthio-5-(3-chloro-1-oxo-2-cyclohexen-2-yl)carbonyl-2,3-dihydrobenzothiophene 1,1-dioxide produced in Example 17-[1] and 16 g of 4-methylbenzenethiol in 5 ml of methylene chloride, 0.20 ml of triethylamine was added dropwise at room temperature. The mixture was stirred at room temperature for one hour. Then, the solvent was distilled away under reduced pressure. The obtained crude product was purified by column chromatography to obtain 0.50 g of the target compound. ¹H-NMR data and IR spectra data of the obtained compound together with the results of measurements of chemical structure and melting point are shown in Table 1.

### EXAMPLE 30

[1] Preparation of 4-chloro-2-methylthio-5-(3-ethylthio-1-oxo-2-cyclohexen-2-yl)carbonyl-2,3-dihydrobenzothiophene 1,1-dioxide (Compound No. 30)

The same procedure as in Example 29-[1] was repeated except that ethanethiol was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 31

[1] Preparation of 4-chloro-2-methylthio-5-[3-(2-hydroxyethyl)thio-1-oxo-2-cyclohexen-2-yl]carbonyl-2,3-dihydrobenzothiophene 1,1-dioxide (Compound No. 31)

The same procedure as in Example 29-[1] was repeated except that 2-hydroxyethanethiol was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 32

[1] Preparation of 4-chloro-2-methylthio-5-[3-(2-cyanoethyl)thio-1-oxo-2-cyclohexen-2-yl]carbonyl-2,3-dihydrobenzothiophene 1,1-dioxide (Compound No. 32)

The same procedure as in Example 29-[1] was repeated except that 2-cyanoethanethiol was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 33

[1] Preparation of 4-chloro-2-methylthio-5-[3-(2-hydroxy-1-propyl)thio-1-oxo-2-cyclohexen-2-yl]carbonyl-2,3-dihydrobenzothiophene 1,1-dioxide (Compound No. 33)

The same procedure as in Example 29-[1] was repeated except that 2-hydroxy-1-propanethiol was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 34

[1] Preparation of 4-chloro-2-methylthio-5-[3-(2-acetoxyethyl)thio-1-oxo-2-cyclohexen-2-yl]carbonyl-2,3-dihydrobenzothiophene 1,1-dioxide (Compound No. 34)

The same procedure as in Example 29-[1] was repeated except that 2-acetoxyethanethiol was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 35

[1] Preparation of 4-chloro-2-methylthio-5-[3-(1-methylimidazol-2-yl)thio-1-oxo-2-cyclohexen-2-yl]carbonyl-2,3-dihydrobenzothiophene 1,1-dioxide (Compound No. 35)

The same procedure as in Example 29-[1] was repeated except that 2-mercapto-1-methylimidazole was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 36

[1] Preparation of 4-chloro-2-(2-methoxyethane)sulfonyl-5-[3-(2-hydroxyethyl)thio-1-oxo-2-cyclohexen-2-yl)carbonyl-2,3-dihydrobenzothiophene 1,1-dioxide (Compound No. 36)

The same procedure as in Example 31-[1] was repeated except that 4-chloro-2-(2-methoxyethane)sulfonyl-5-(3-chloro-1-oxo-2-cyclohexen-2-yl)carbonyl-2,3-dihydrobenzothiophene 1,1-dioxide was used in place of 4-chloro-2-methylthio-5-(3-chloro-1-oxo-2-cyclohexen-2-yl)carbonyl-2,3-dihydrobenzothiophene 1, 1-dioxide, thereby obtaining the target compound. ¹H-NMR data and IR spectra data of the obtained compound together with the results of measurements of chemical structure and melting point are shown in Table 1.

### EXAMPLE 37

[1] Preparation of 4-chloro-2-methylthio-5-(3-ethoxy-1-oxo-2-cyclohexen-2-yl)carbonyl-2,3-dihydrobenzothiophene 1,1-dioxide (Compound No. 37)

After dissolving 1.0 g of 4-chloro-2-methylthio-5-(1,3-dioxocyclohex-2-yl)carbonyl-2,3-dihydrobenzothiophene 1,1-dioxide produced in Example 1-[2] in 10 ml of methylene chloride, 0.52 ml of diethylaminosulfur trifluoride was added dropwise with ice-cooling. The mixture was stirred at room temperature for one hour. Thereafter, the resultant reaction solution was washed with an aqueous sodium hydrogen carbonate solution, and then dried over anhydrous sodium sulfate. Then, the solvent was distilled away under reduced pressure, and the resultant crude product was recrystallized from methanol to obtain 0.62 g of the target compound. ¹H-NMR data and IR spectra data of the obtained compound together with the results of measurements of chemical structure and melting point are shown in Table 1.

### EXAMPLE 38

[1] Preparation of 4-chloro-2-methylthio-5-[3-(4,5-dihydropyrazol-1-yl)-1-oxo-2-cyclohexen-2-yl]carbonyl-2,3-dihydrobenzothiophene 1,1-dioxide (Compound No. 38)

After dissolving 0.50 g of 4-chloro-2-methylthio-5-(3-chloro-1-oxo-2-cyclohexen-2-yl)carbonyl-2,3-dihydrobenzothiophene 1,1-dioxide produced in Example 17-[1] in 5 ml of methylene chloride, 0.18 g of 4,5-dihydropyrazole was added at room temperature. The mixture was stirred at room temperature for one hour. Then, the solvent was distilled away under reduced pressure, and the resultant crude product was purified by column chromatography to obtain 0.39 g of the target compound. ¹H-NMR data and IR spectra data of the obtained compound together with the results of measurements of chemical structure and melting point are shown in Table 1.

### EXAMPLE 39

[1] Preparation of 4-chloro-2-methylthio-5-[3-(pyrazol-1-yl)-1-oxo-2-cyclohexen-2-yl]carbonyl-2,3-dihydrobenzothiophene 1,1-dioxide (Compound No. 39)

The same procedure as in Example 38-[1] was repeated except that pyrazole was used in place of 4,5-dihydropyrazole, thereby obtaining the target compound. ¹H-NMR data and IR spectra data of the obtained compound together with the results of measurements of chemical structure and melting point are shown in Table 1.

### EXAMPLE 40

[1] Preparation of 4-chloro-2-methylthio-5-[3-(bis-methoxycarbonyl)methyl-1-oxo-2-cyclohexen-2-yl]carbonyl-2,3-dihydrobenzothiophene 1,1-dioxide (Compound No. 40)

After suspending 0.15 g of 60 wt % sodium hydride in 8 ml of benzene, 0.49 g of dimethyl malonate was added at room temperature. The mixture was stirred at room temperature for 30 min. Then, the mixture was added with 0.50 g of 4-chloro-2-methylthio-5-(3-chloro-1-oxo-2-cyclohexen-2-yl)carbonyl-2,3-dihydrobenzothiophene 1,1-dioxide produced in Example 17-[1], and refluxed under heating for 2 h. The resultant reaction solution was added with ethyl acetate, washed with a 5% hydrochloric acid, and then dried over anhydrous sodium sulfate. Then, the solvent was distilled away under reduced pressure, and the resultant crude product was purified by column chromatography to obtain 0.43 g of the target compound. ¹H-NMR data and IR spectra data of the obtained compound together with the results of measurements of chemical structure and melting point are shown in Table 1.

### EXAMPLE 41

[1] Preparation of 4-bromo-2-methylthio-5-(1,3-dioxocyclohex-2-yl)carbonyl-2,3-dihydrobenzothiophene 1,1-dioxide (Compound No. 41)

The same procedure as in Example 1-[2] was repeated except that 4-bromo-5-oxycarbonyl-2-methylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 42

[1] Preparation of 4-iodo-2-methylthio-5-(1,3-dioxocyclohex-2-yl)carbonyl-2,3-dihydrobenzothiophene 1,1-dioxide (Compound No. 42)

The same procedure as in Example 1-[2] was repeated except that 4-iodo-5-oxycarbonyl-2-methylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 43

[1] Preparation of 4-chloro-7-methyl-2-methylthio-5-(1,3-dioxocyclohex-2-yl)carbonyl-2,3-dihydrobenzothiophene 1,1-dioxide (Compound No. 43)

The same procedure as in Example 1-[2] was repeated except that 4-chloro-7-methyl-5-oxycarbonyl-2-methylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 44

[1] Preparation of 4-chloro-7-methyl-2-ethylthio-5-(1,3-dioxocyclohex-2-yl)carbonyl-2,3-dihydrobenzothiophene 1,1-dioxide (Compound No. 44)

The same procedure as in Example 1-[2] was repeated except that 4-chloro-7-methyl-5-oxycarbonyl-2-ethylthio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 45

[1] Preparation of 4-chloro-7-methyl-2-(2-methoxyethyl)thio-5-(1,3-dioxocyclohex-2-yl)carbonyl-2, 3-dihydrobenzothiophene 1,1-dioxide (Compound No. 45)

The same procedure as in Example 1-[2] was repeated except that 4-chloro-7-methyl-5-oxycarbonyl-2-(2-methoxyethyl)thio-2,3-dihydrobenzothiophene 1,1-dioxide was used in place of 4-chloro-5-oxycarbonyl-2-methylthio-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 46

[1] Preparation of 4-chloro-7-methyl-2-methanesulfonyl-5-(1,3-dioxocyclohex-2-yl)carbonyl-2,3-dihydrobenzothiophene 1,1-dioxide (Compound No. 46)

The same procedure as in Example 15-[1] was repeated except that 4-chloro-7-methyl-2-methylthio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 47

[1] Preparation of 4-chloro-7-methyl-2-ethanesulfonyl-5-(1,3-dioxocyclohex-2-yl)carbonyl-2,3-dihydrobenzothiophene 1,1-dioxide (Compound No. 47)

The same procedure as in Example 15-[1] was repeated except that 4-chloro-7-methyl-2-ethylthio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 48

[1] Preparation of 4-chloro-7-methyl-2-(2-methoxyethane)sulfonyl-5-(1,3-dioxocyclohex-2-yl)carbonyl-2,3-dihydrobenzothiophene 1,1-dioxide (Compound No. 48)

The same procedure as in Example 15-[1] was repeated except that 4-chloro-7-methyl-2-(2-methoxyethyl)thio-5-(1,3-dioxocyclohex-2-yl)carbonyl-2, 3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 49

[1] Preparation of 4-chloro-7-methyl-2-methylthio-5-(3-chloro-1-oxo-2-cyclohexen-2-yl)carbonyl-2, 3-dihydrobenzothiophene 1,1-dioxide (Compound No. 49)

The same procedure as in Example 17-[1] was repeated except that 4-chloro-7-methyl-2-methylthio-5-(1,3-dioxocyclohex-2-yl)carbonyl-2,3-dihydrobenzothiophene 1,1-dioxide was used in place of 4-chloro-2-methylthio-5-(1,3-dioxocyclohex-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE 50

[1] Preparation of 4-chloro-7-methyl-2-methylthio-5-[3-(2-hydroxyethyl)thio-1-oxo-2-cyclohexen-2-yl]carbonyl-2,3-dihydrobenzothiophene 1,1-dioxide (Compound No. 50)

The same procedure as in Example 31-[1] was repeated except that 4-chloro-7-methyl-2-methylthio-5-(3-chloro-1-oxo-2-cyclohexen-2-yl)carbonyl-2, 3-dihydrobenzothiophene 1,1-dioxide was used in place of 4-chloro-2-methylthio-5-(3-chloro-1-oxo-2-cyclohexen-2-yl)carbonyl-2,3-diliydrobenzothiophene 1,1-dioxide, thereby obtaining the target compound. ¹H-NMR data and IR spectra data of the obtained compound together with the results of measurements of chemical structure and melting point are shown in Table 1.

### EXAMPLE51

[1] Preparation of 4-chloro-7-methyl-2-methylthio-5-[3-(2-chloroacetoxyethyl)thio-1-oxo-2-cyclohexen-2-yl]carbonyl-2,3-dihydrobenzothiophene 1,1-dioxide (Compound No. 51)

The same procedure as in Example 29-[1] was repeated except that 4-chloro-7-methyl-2-methylthio-5-(3-chloro-1-oxo-2-cyclohexen-2-yl)carbonyl-2, 3-dihydrobenzothiophene 1,1-dioxide was used in place of 4-chloro-2-methylthio-5-(3-chloro-1-oxo-2-cyclohexen-2-yl)carbonyl-2,3-dihydrobenzothiophene 1,1-dioxide, and 2-chloroacetoxyethanethiol was used in place of 4-methylbenzenethiol, thereby obtaining the target compound. ¹H-NMR data and IR spectra data of the obtained compound together with the results of measurements of chemical structure and melting point are shown in Table 1.

### EXAMPLE 52

[1] Preparation of 4-chloro-7-methyl-2-methylthio-5-[3-(pyrazol-1-yl) -1-oxo-2-cyclohexen-2-yl]carbonyl-2,3-dihydrobenzothiophene 1,1-dioxide (Compound No. 52)

The same procedure as in Example 39-[1] was repeated except that 4-chloro-7-methyl-2-methylthio-5-(3-chloro-1-oxo-2-cyclohexen-2-yl)carbonyl-2, 3-dihydrobenzothiophene 1,1-dioxide was used in place of 4-chloro-2-methylthio-5-(3-chloro-1-oxo-2-cyclohexen-2-yl)carbonyl-2,3-dihydrobenzothiophene 1,1-dioxide, thereby obtaining the target compound. ¹H-NMR data and IR spectra data of the obtained compound together with the results of measurements of chemical structure and melting point are shown in Table 1.

TABLE 1

| | | NMR ppm(CDCl₃, | IR | mp |
|---|---|---|---|---|
| | Chemical Structure | TMS Standard) | cm⁻¹ | (° C.) |
| 1 | 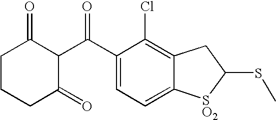 | 1.9-2.2(2H, m) 2.3-2.6(2H, m) 2.44(3H, s) 2.7-2.9(2H, m) 3.06(1H, dd) 3.71(1H, dd) 4.42(1H, dd) 7.29(1H, d) 7.73(1H, d) | 1662 1558 1542 1306 1152 1137 | 174.7-175.1 |
| 2 | 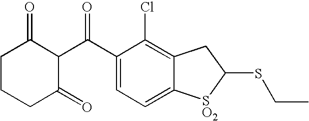 | 1.39(3H, t) 1.9-2.3(2H, m) 2.3-2.6(2H, m) 2.7-3.2(5H, m) 3.72(1H, dd) 4.49(1H, dd) 7.30(1H, d) 7.74(1H, d) | 1674 1595 1545 1309 1148 1125 | — |
| 3 | 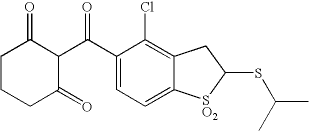 | 1.41(3H, d) 1.45(3H, d) 1.9-2.3(2H, m) 2.3-2.6(2H, m) 2.7-3.2(3H, m) 3.47(1H, qq) 3.71(1H, dd) 4.54(1H, dd) 7.30(1H, d) 7.73(1H, d) | 1673 1558 1403 1313 1150 1135 | — |
| 4 | 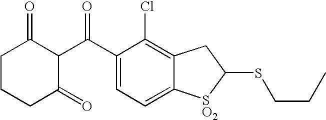 | 1.06(3H, t) 1.5-1.9(2H, m) 1.9-2.3(2H, m) 2.3-2.6(2H, m) 2.6-3.2(5H, m) 3.72(1H, dd) 4.47(1H, dd) 7.31(1H, d) 7.74(1H, d) | 1673 1558 1403 1313 1151 1135 | — |
| 5 | 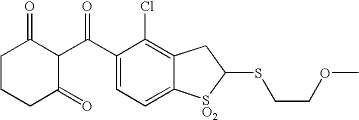 | 1.9-2.3(2H, m) 2.3-2.6(2H, m) 2.7-3.9(8H, m) 3.41(3H, s) 4.73(1H, dd) 7.29(1H, d) 7.73(1H, d) | 1673 1557 1403 1312 1151 1135 | — |
| 6 | 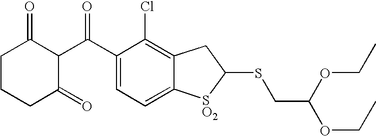 | 1.24(3H, t) 1.25(3H, t) 1.9-2.2(2H, m) 2.3-2.6(2H, m) 2.7-2.9(2H, m) 2.9-3.9(4H, m) 3.63(4H, q) 4.6-4.9(2H, m) 7.29(1Hd) 7.72(1H, d) | 1674 1557 1403 1313 1151 1135 1056 | — |
| 7 | 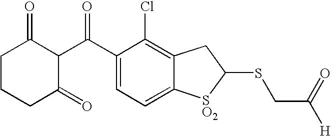 | 1.9-2.3(2H, m) 2.3-2.7(2H, m) 2.7-3.0(2H, m) 3.07(1H, dd) 3.4-4.1(3H, m) 4.51(1H, dd) 7.31(1H, d) 7.73(1H, d) 9.75(1H, s) | 1672 1557 1403 1312 1151 1136 | — |
| 8 | 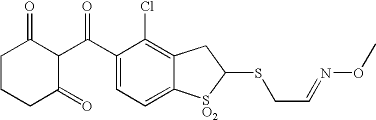 | Mixture of regio isomer 1.9-2.3(2H) 2.3-2.6(2H) 2.7-3.0(2H) 3.0-4.0(7H) 4.4-4.6(1H) 6.8-7.8(3H) | 1673 1558 1403 1313 1152 1135 | — |
| 9 | 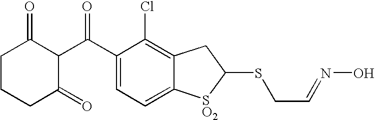 | Mixture of regio isomer 1.9-2.3(2H) 2.3-2.6(2H) 2.7-3.0(2H) 3.0-4.0(4H) 4.4-4.6(1H) 6.8-7.8(3H) | 1672 1556 1403 1312 1151 1135 | — |
| 10 | 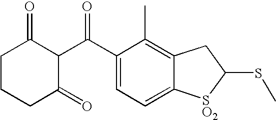 | 1.9-2.2(2H, m) 2.17(3H, s) 2.3-2.6(2H, m) 2.46(3H, s) 2.7-2.9(2H, m) 2.99(1H, dd) 3.59(1H, dd) 4.39(1H, dd) 7.16(1H, d) 7.63(1H, d) | 1662 1541 1434 1406 1301 1188 1150 1125 | 192.7-193.2 |
| 11 | 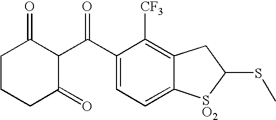 | 1.9-2.2(2H, m) 2.3-2.6(2H, m) 2.46(3H, s) 2.7-2.9(2H, m) 3.27(1H, dd) 3.86(1H, dd) 4.42(1H, dd) 7.23(1H, d) 7.94(1H, d) | 1664 1552 1309 1163 1121 | 206.1-211.6 |
| 12 | 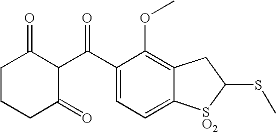 | 1.9-2.3(2H, m) 2.3-2.6(2H, m) 2.45(3H, s) 2.7-2.9(2H, m) 3.05(3H, dd) 3.70(1H, dd) 3.76(3H, s) 4.37(1H, dd) 7.23(1H, d) 7.51(1H, d) | 1667 1586 1550 1411 1300 1195 1123 | — |
| 13 | 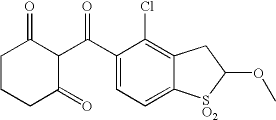 | 1.9-2.3(2H, m) 2.3-2.6(2H, m) 2.7-2.9(2H, m) 3.19(1H, dd) 3.62(1H, dd) 3.81(3H, s) 4.80(1H, dd) 7.28(1H, d) 7.71(1H, d) | 1676 1564 1408 1296 1194 1142 1117 | — |
| 14 | 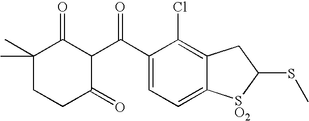 | 1.12(3H, s) 1.35(3H, s) 1.8-2.0(2H, m) 2.46(3H, s) 2.7-3.0(2H, m) 3.05(1H, dd) 3.71(1H, dd) 4.42(1H, dd) 7.29(1H, d) 7.75(1H, d) | 1672 1559 1405 1311 1152 1136 | — |
| 15 | 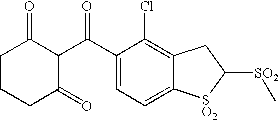 | 1.9-2.3(2H, m) 2.3-2.6(2H, m) 2.7-3.0(2H, m) 3.31(3H, s) 3.81(2H, d) 4.81(1H, dd) 7.33(1H, d) 7.72(1H, d) | 1671 1558 1404 1327 1162 1143 1124 | — |
| 16 | 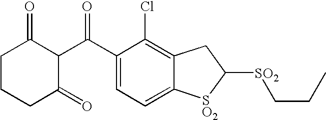 | 1.15(3H, t) 1.8-2.3(2H, m) 2.3-2.6(2H, m) 2.7-3.0(2H, m) 3.3-3.6(2H, m) 3.7-3.9(2H, m) 4.80(1H, dd) 7.33(1H, d) 7.70(1H, d) | 1672 1558 1405 1329 1163 1137 | — |
| 17 | 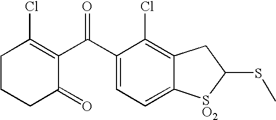 | 2.0-2.3(2H, m) 2.4-2.7(2H, m) 2.45(3H, s) 2.8-3.0(2H, m) 3.07(1H, dd) 3.74(1H, dd) 4.42(1H, dd) 7.74(1H, d) 7.83(1H, d) | 1697 1670 1310 1286 1152 1137 | — |
| 18 | 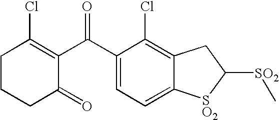 | 2.0-2.4(2H, m) 2.4-2.7(2H, m) 2.8-3.1(2H, m) 3.39(3H, s) 3.89(2H, d) 5.31(1H, dd) 7.77(1H, d) 7.92(1H, d) [acetone · d⁶] | 1702 1663 1323 1292 1280 1171 1136 | — |
| 19 | 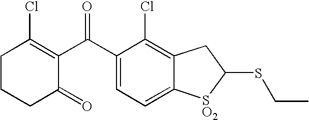 | 1.39(3H, t) 2.0-2.4(2H, m) 2.4-2.7(2H, m) 2.7-3.2(5H, m) 3.77(1H, dd) 4.50(1H, dd) 7.78(2H, s) | 1671 1314 1285 1151 1136 | — |
| 20 | 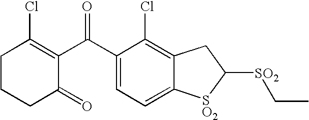 | 1.45(3H, t) 2.1-2.3(2H, m) 2.3-2.6(2H, m) 2.8-3.1(2H, m) 3.59(2H, q) 3.8-4.0(2H, m) 5.50(1H, dd) 7.86(1H, d) 8.00(1H, d) [acetone · d⁶] | 1704 1662 1328 1294 1281 1169 | — |
| 21 | 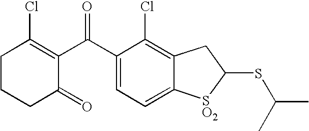 | 1.41(3H, d) 1.44(3H, d) 2.0-2.4(2H, m) 2.4-2.7(2H, m) 2.7-3.2(3H, m) 3.46(1H, qq) 3.75(1H, dd) 4.54(1H, dd) 7.78(2H, s) | 1697 1672 1315 1285 1150 1136 | — |
| 22 | 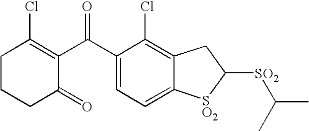 | 1.49(3H, d) 1.57(3H, d) 2.0-2.4(2H, m) 2.4-2.7(2H, m) 2.8-3.0(2H, m) 3.5-4.2(3H, m) 4.91(1H, dd) 7.69(1H, d) 7.86(1H, d) | 1699 1671 1331 1286 1174 1133 | — |
| 23 | 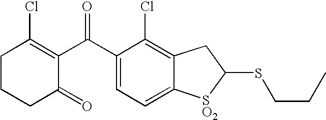 | 1.04(3H, t) 1.5-1.9(2H, m) 2.0-2.4(2H, m) 2.4-2.7(2H, m) 2.7-3.2(5H, m) 3.74(1H, dd) 4.48(1H, dd) 7.78(2H, s) | 1696 1672 1315 1285 1151 1136 | — |
| 24 | 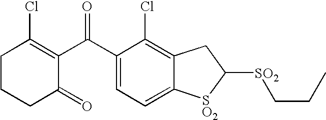 | 1.16(3H, t) 1.8-2.4(4H, m) 2.4-2.7(2H, m) 2.7-3.0(2H, m) 3.3-3.7(2H, m) 3.7-4.0(2H, m) 4.77(1H, dd) 7.72(1H, d) 7.87(1H, d) | 1697 1671 1330 1288 1182 1137 | — |
| 25 | 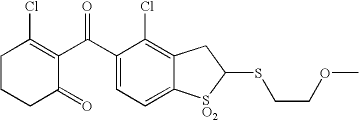 | 2.0-2.4(2H, m) 2.4-2.7(2H, m) 2.7-3.3(5H, m) 3.3-4.0(3H, m) 3.39(3H, s) 4.75(1H, dd) 7.71(1H, d) 7.82(1H, d) | 1673 1315 1286 1151 1136 1117 | — |
| 26 | 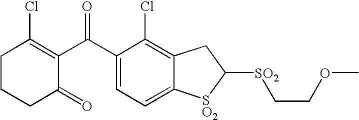 | 2.0-2.4(2H, m) 2.4-2.7(2H, m) 2.7-3.0(2H, m) 3.2-4.0(5H, m) 3.39(3H, s) 4.32(1H, dd) 5.52(1H, dd) 7.76(1H, d) 7.92(1H, d) | 1698 1671 1333 1288 1181 1137 1115 | — |
| 27 | 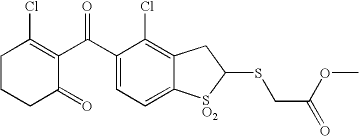 | 2.0-2.4(2H, m) 2.4-2.7(2H, m) 2.8-3.0(2H, m) 3.09(1H, dd) 3.3-4.0(3H, m) 3.80(3H, s) 7.83(1H, dd) 7.73(1H, d) 7.84(1H, d) | 1698 1671 1314 1287 1151 1137 | — |
| 28 | 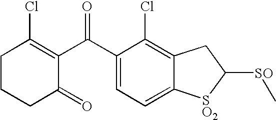 | Mixture of regio isomer 2.0-2.4(2H) 2.4-2.7(2H) 2.8-3.0(2H) 2.99(3H) 3.5-4.2(2H) 4.4-4.9(2H) 7.6-7.9(2H) | 1694 1670 1319 1287 1181 1138 1064 | — |
| 29 | 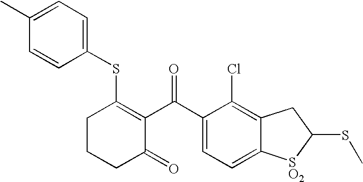 | 1.8-2.1(2H, m) 2.3-2.6(4H, m) 2.41(3H, s) 2.45(3H, s) 3.06(1H, dd) 3.71(1H, dd) 4.41(1H, dd) 7.25(2H, d) 7.43(2H, d) 7.54(1H, d) 7.74(1H, d) | 1657 1519 1344 1306 1294 1134 | — |
| 30 | 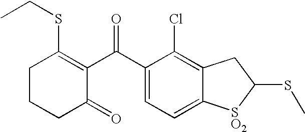 | 1.37(3H, t) 2.0-2.3(2H, m) 2.3-2.6(2H, m) 2.43(3H, s) 2.8-3.2(5H, m) 3.70(1H, dd) 4.41(1H, dd) 7.48(1H, d) 7.72(1H, d) | 1653 1345 1307 1151 1136 | — |
| 31 | 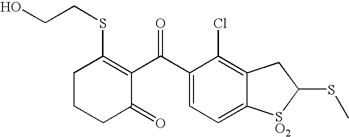 | 1.7-2.3(4H, m) 2.3-3.3(5H, m) 2.44(3H, s) 3.5-4.0(3H, m) 4.41(1H, dd) 7.54(1H, d) 7.66(1H, d) | 1652 1398 1346 1305 1151 1136 | — |
| 32 | 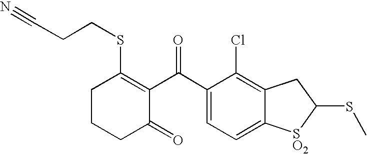 | 1.8-3.4(11H, m) 2.42(3H, s) 3.71(1H, dd) 4.41(1H, dd) 7.52(1H, d) 7.72(1H, d) | 1657 1398 1345 1305 1189 1150 1136 | — |
| 33 | 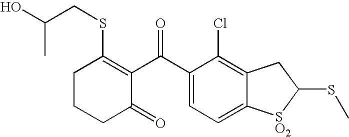 | 1.33(3H, d) 1.8-2.8(5H, m) 2.44(3H, s) 2.8-3.3(4H, m) 3.70(1H, dd) 3.8-4.2(1H, m) 4.41(1H, dd) 7.53(1H, d) 7.72(1H, d) | 1647 1398 1345 1305 1188 1150 1135 | — |
| 34 | 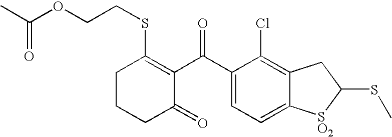 | 2.0-2.3(2H, m) 2.20(3H, s) 2.4-2.6(2H, m) 2.44(3H, s) 2.8-3.3(3H, m) 3.19(2H, t) 3.70(1H, dd) 4.28(2H, t) 4.34(1H, dd) 7.52(1H, d) 7.72(1H, d) | 1738 1655 1307 1227 1151 1137 | — |
| 35 | 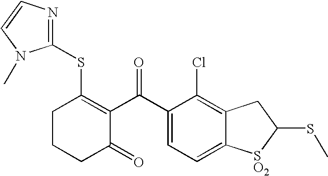 | 1.9-2.2(2H, m) 2.3-2.6(4H, m) 2.45(3H, s) 3.04(1H, dd) 3.71(1H, dd) 3.79(3H, s) 4.41(1H, dd) 7.15(1H, d) 7.25(1H, d) 7.54(1H, d) 7.74(1H, d) | 1667 1308 1280 1151 1136 | — |
| 36 | 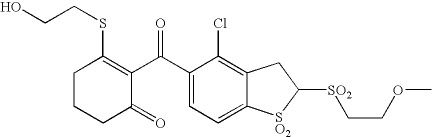 | 1.6-2.3(2H, m) 2.3-2.9(4H, m) 2.9-3.4(2H, m) 3.4-4.6(8H, m) 3.47(3H, s) 5.15(1H, dd) 7.58(1H, d) 7.63(1H, d) | 1559 1401 1331 1279 1180 1115 | — |
| 37 | 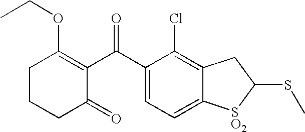 | 1.19(3H, t) 1.9-2.3(2H, m) 2.3-2.6(2H, m) 2.44(3H, s) 2.6-2.8(2H, m) 3.05(1H, dd) 3.73(1H, dd) 4.08(2H, q) 4.42(1H, dd) 7.60(1H, d) 7.72(1H, d) | 1686 1567 1407 1387 1303 1152 1133 1036 | — |
| 38 | 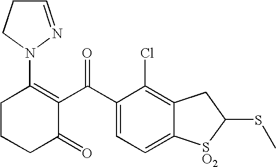 | 1.8-2.2(2H, m) 2.2-2.5(2H, m) 2.43(3H, s) 2.8-3.3(5H, m) 3.5-3.9(3H, m) 4.39(1H, dd) 7.3-7.4(1H, m) 7.54(1H, d) 7.68(1H, d) | 1546 1504 1435 1304 1150 1135 | — |
| 39 | 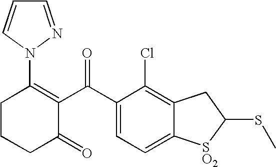 | 2.1-2.8(4H, m) 2.45(3H, s) 2.9-3.2(3H, m) 3.77(1H, dd) 4.40(1H, dd) 6.43(1H, dd) 7.52(1H, d) 7.68(1H, d) 7.76(1H, d) 7.99(1H, d) | 1667 1647 1622 1371 1301 1140 | — |
| 40 | 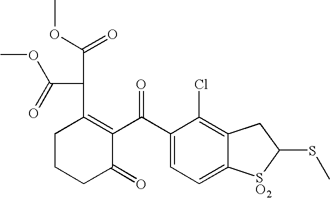 | 2.0-2.3(2H, m) 2.4-2.7(2H, m) 2.44(3H, s) 2.7-2.9(2H, m) 3.04(1H, dd) 3.72(1H, dd) 3.81(6H, s) 4.41(1H, dd) 4.79(1H, s) 7.71(2H, s) | 1754 1743 1676 1302 1288 1147 1133 | 150.3-150.5 |
| 41 | 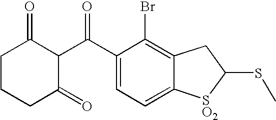 | 1.9-2.3(2H, m) 2.3-2.6(2H, m) 2.45(3H, s) 2.7-2.9(2H, m) 3.04(1H, dd) 3.68(1H, dd) 4.42(1H, dd) 7.24(1H, d) 7.77(1H, d) | 1664 1558 1549 1307 1147 1135 | — |
| 42 | 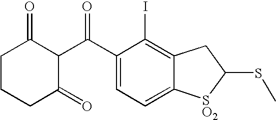 | 1.9-2.3(2H, m) 2.3-3.2(5H, m) 2.44(3H, s) 3.59(1H, dd) 4.43(1H, dd) 7.17(1H, d) 7.79(1H, d) | 1664 1584 1554 1305 1184 1147 1135 | — |
| 43 | 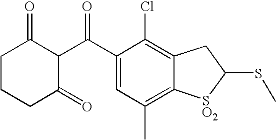 | 1.9-2.3(2H, m) 2.3-2.6(2H, m) 2.46(3H, s) 2.63(3H, s) 2.7-2.9(2H, m) 2.99(1H, dd) 3.68(1H, dd) 4.40(1H, dd) 7.05(1H, s) | 1658 1544 1430 1300 1191 1138 | 195.5-197.0 |
| 44 | 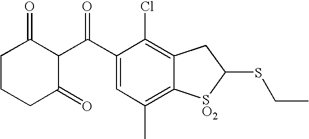 | 1.39(3H, t) 1.9-2.3(2H, m) 2.3-2.6(2H, m) 2.62(3H, s) 2.7-3.2(5H, m) 3.61(1H, dd) 4.47(1H, dd) 7.03(1H, s) | 1673 1558 1552 1302 1136 | 143.2-154.8 |
| 45 | 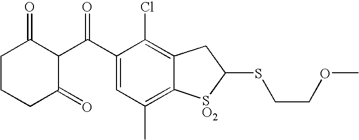 | 1.9-2.3(2H, m) 2.3-3.4(7H, m) 2.62(3H, s) 3.42(3H, s) 3.5-3.9(3H, m) 4.69(1H, dd) 7.03(1H, s) | 1674 1558 1419 1375 1309 1137 | — |
| 46 | 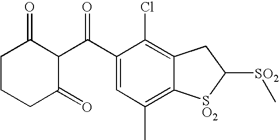 | 1.9-2.3(2H, m) 2.3-2.7(2H, m) 2.61(3H, s) 2.7-2.9(2H, m) 3.33(3H, s) 3.76(2H, d) 4.74(1H, dd) 7.08(1H, s) | 1674 1558 1329 1166 1148 1134 | — |
| 47 | 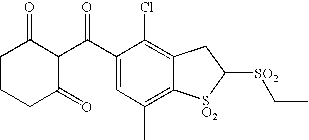 | 1.56(3H, t) 1.9-2.3(2H, m) 2.3-2.6(2H, m) 2.60(3H, s) 2.7-2.9(2H, m) 3.3-3.9(4H, m) 4.77(1H, dd) 7.08(1H, s) | 1674 1557 1456 1418 1329 1136 | — |
| 48 | 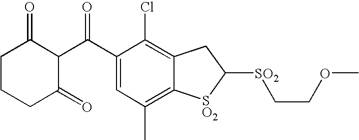 | 1.9-2.3(2H, m) 2.4-2.7(2H, m) 2.60(3H, s) 2.7-2.9(2H, m) 3.1-4.5(6H, m) 3.47(3H, s) 5.10(1H, dd) 7.06(1H, s) | 1673 1557 1330 1178 1126 | — |
| 49 | 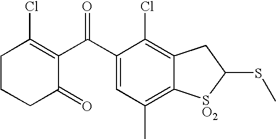 | 2.0-2.4(2H, m) 2.4-2.7(2H, m) 2.45(3H, s) 2.64(3H, s) 2.8-3.2(3H, m) 3.69(1H, dd) 4.40(1H, dd) 7.55(1H, s) | 1673 1300 1273 1187 1156 1128 | — |
| 50 | 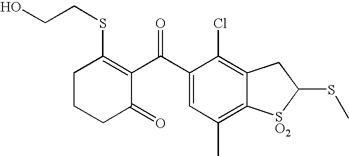 | 2.0-2.3(2H, m) 2.4-2.7(2H, m) 2.44(3H, s) 2.62(3H, s) 2.8-3.2(3H, m) 3.14(2H, t) 3.65(1H, dd) 3.90(2H, t) 4.37(1H, dd) 7.29(1H, s) | 1648 1456 1436 1346 1302 1131 | — |
| 51 | 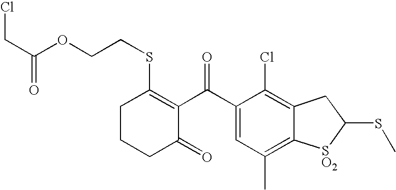 | 2.0-2.3(2H, m) 2.3-2.7(2H, m) 2.44(3H, s) 2.62(3H, s) 2.7-3.2(3H, m) 3.22(2H, t) 3.65(1H, dd) 4.10(2H, s) 4.38(1H, dd) 4.40(2H, t) 7.27(1H, s) | 1753 1655 1345 1303 1131 | — |
| 52 | 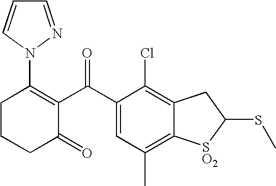 | 2.2-2.8(4H, m) 2.44(3H, s) 2.62(3H, s) 2.8-3.2(3H, m) 3.70(1H, dd) 4.38(1H, dd) 6.44(1H, dd) 7.53(1H, d) 7.75(1H, s) 7.76(1H, d) | 1670 1628 1373 1297 1149 1131 | — |

### WEED CONTROL EXAMPLE

[1] Preparation of Herbicidal Composition

A mixture consisting of 57 parts by weight of talc and 40 parts by weight of bentonite as carrier and 3 parts by weight of sodium alkylbenzenesulfonate as surfactant was pulverized and mixed uniformly to obtain a carrier for wettable powder. Each herbicidal composition was prepared by uniformly pulverizing and mixing 90 parts by weight of the carrier for wettable powder and 10 parts by weight of the benzothiophene derivative produced in each example.

[2] Weed Control Test

(1) Submersion Test/treatment 3 Days after Transplantation

Seeds of Scirpus juncoides were sowed onto paddy field soil in a Wagner pot of 1/2000 are. Then, rice seedlings of 2.5 leaf stage were transplanted in the soil. Water was pored into the pot so as to submerge the soil at a depth of 3 cm. The pot was placed in a greenhouse maintained at 20 to 25° C. for vegetation. After 3 days from transplantation of the rice seedlings, a predetermined amount of the herbicidal composition prepared in the step [1] was applied to the pot. After 30 days from the application of the herbicidal composition, the herbicidal effect and injury to the paddy rice were determined. The results are shown in Table 2.

(1) Submersion Test/treatment 10 Days after Transplantation

Seeds of Scirpus juncoides were sowed onto paddy field soil in a Wagner pot of 1/2000 are. Then, rice seedlings of 2.5 leaf stage were transplanted in the soil. Water was pored into the pot so as to submerge the soil at a depth of 3 cm. The pot was placed in a greenhouse maintained at 20 to 25° C. for vegetation. After 10 days from transplantation of the rice seedlings, a predetermined amount of the herbicidal composition prepared in the step [1] was applied to the pot. After 30 days from the application of the herbicidal composition, the herbicidal effect and injury to the paddy rice were determined. The results are shown in Table 2.

The herbicidal effect and the injury to crops in the above tests (1) and (2) were evaluated based on the following ratings.

(a) Degree of Weed Control

The degree of weed control (%) was calculated from the weights of the above-ground parts of fresh weeds in the treated area and the untreated area using the following equation:

Degree of weed control (%)=[1−(weight of weeds in treated area)/(weight of weeds in untreated area)]×100

(b) Herbicidal Effect

The herbicidal effect was determined according to the following ratings:

| Herbicidal Effect | Degree of weed Control |
|---|---|
| 0 | less than 5% (almost no herbicidal effect) |
| 1 | 5% or higher but less than 20% |
| 2 | 20% or higher but less than 40% |
| 3 | 40% or higher but less than 70% |
| 4 | 70% or higher but less than 90% |
| 5 | 90% or higher (almost completely dead) |

(c) Injury to Crops

The injury to crops was evaluated according to the following ratings:

| Injury to Crops | Degree of phytotoxicity |
|---|---|
| 0 | No injury |
| 1 | Substantially no injury |
| 2 | Slight injury |
| 3 | Some injury |
| 4 | Remarkable injury |
| 5 | Almost all crops dead |

TABLE 2

| | | Treatment 3 | | Treatment 10 |
|---|---|---|---|---|
| | | days after | | days after |
| | | transplantation | | transplantation |
| | Appli- | Herbicidal | | Herbicidal |
| Com- | cation | effect | Injury | effect |
| pound | rate | Scirpus | paddy | Scirpus |
| No. | (g/ha) | juncoides | rice | juncoides |
| 1 | 100 | 5 | 0 | 5 |
| | 200 | 5 | 0 | 5 |
| 2 | 100 | 5 | 0 | 5 |
| | 200 | 5 | 0 | 5 |
| 5 | 100 | 5 | 0 | 5 |
| | 200 | 5 | 0 | 5 |
| 8 | 100 | 4 | 0 | 5 |
| | 200 | 5 | 0 | 5 |
| 10 | 100 | 5 | 0 | 5 |
| | 200 | 5 | 0 | 5 |
| 11 | 100 | 5 | 0 | 5 |
| | 200 | 5 | 0 | 5 |
| 15 | 100 | 5 | 0 | 4 |
| | 200 | 5 | 0 | 5 |
| 17 | 100 | 5 | 0 | 5 |
| | 200 | 5 | 0 | 5 |
| 18 | 100 | 4 | 0 | 4 |
| | 200 | 5 | 0 | 5 |
| 19 | 100 | 5 | 0 | 4 |
| | 200 | 5 | 0 | 5 |
| 25 | 100 | 4 | 0 | 4 |
| | 200 | 5 | 0 | 5 |
| 27 | 100 | 4 | 0 | 4 |
| | 200 | 5 | 0 | 5 |
| 28 | 100 | 5 | 0 | 5 |
| | 200 | 5 | 0 | 5 |
| 31 | 100 | 5 | 0 | 5 |
| | 200 | 5 | 0 | 5 |
| 33 | 100 | 4 | 0 | 4 |
| | 200 | 5 | 0 | 5 |
| 34 | 100 | 4 | 0 | 4 |
| | 200 | 5 | 0 | 5 |
| 35 | 100 | 4 | 0 | 4 |
| | 200 | 5 | 0 | 5 |
| 37 | 100 | 5 | 0 | 5 |
| | 200 | 5 | 0 | 5 |
| 38 | 100 | 4 | 0 | 4 |
| | 200 | 5 | 0 | 5 |
| 39 | 100 | 4 | 0 | 4 |
| | 200 | 5 | 0 | 5 |
| 40 | 100 | 4 | 0 | 4 |
| | 200 | 5 | 0 | 5 |
| 41 | 100 | 5 | 0 | 5 |
| | 200 | 5 | 0 | 5 |
| 43 | 100 | 5 | 0 | 5 |
| | 200 | 5 | 0 | 5 |
| 44 | 100 | 5 | 0 | 5 |
| | 200 | 5 | 0 | 5 |
| 45 | 100 | 5 | 0 | 5 |
| | 200 | 5 | 0 | 5 |
| 46 | 100 | 5 | 0 | 4 |
| | 200 | 5 | 0 | 5 |
| 49 | 100 | 5 | 0 | 5 |
| | 200 | 5 | 0 | 5 |
| 50 | 100 | 5 | 0 | 5 |
| | 200 | 5 | 0 | 5 |
| 51 | 100 | 4 | 0 | 4 |
| | 200 | 5 | 0 | 5 |
| 52 | 100 | 4 | 0 | 4 |
| | 200 | 5 | 0 | 5 |

### WEED CONTROL COMPARATIVE EXAMPLE

The same procedure as in Weed Control Example was repeated except for using the compound (A) disclosed in WO 00/20408 and the compound (B) (Development No. SB-500 available from S.D.S. Biotec Co., Ltd.) in place of the benzothiophene derivatives of the present invention. The results are showin 3.

TABLE 3

| | | Treatment 3 | | Treatment 10 |
|---|---|---|---|---|
| | | days after | | days after |
| | | transplantation | | transplantation |
| | Appli- | Herbicidal | | Herbicidal |
| Com- | cation | effect | Injury | effect |
| pound | rate | Scirpus | paddy | Scirpus |
| No. | (g/ha) | juncoides | rice | juncoides |
| (A) | 100 | 4 | 0 | 4 |
| | 200 | 5 | 1 | 4 |
| (B) | 100 | 3 | 0 | 2 |
| | 200 | 4 | 0 | 3 |

Industrial Apiplicability

The herbicide composition containing the benzothiophene derivative of the present invention as a herbicidally effective ingredient is less injury to useful crops such as paddy rice, and has a broad spectrum of controlling the growth of weeds in a low application rate.

The herbicidal composition containing the benzothiophene derivative of the present invention is especially suitable for use in paddy rice field.

1. A benzothiophene derivative represented by the following formula (I): 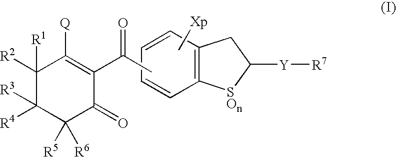
wherein:
each of R¹ to R⁶ independently represents hydrogen, halogen, C₁-C₆ alkyl or C₁-C₆ haloalkyl, and any two of R¹ to R⁶ may be bonded to each other to form a bicyclic ring structure together with a cyclohexene ring;
R⁷ represents C₁-C₆ alkyl which may be substituted;
Q represents hydroxyl or halogen; or Q represents C₁-C₆ alkoxyl, C₁-C₆ alkylthio, C₁-C₆ alkylsulfinyl, C₁-C₆ alkylsulfonyl, phenoxy, phenylthio, phenylsulfinyl, phenylsulfonyl, C₂-C₁₂ dialkylamino, C₂-C₁₂ N-alkoxyalkylamino, five- or six-membered nitrogen-containing heterocyclic residue which can be bonded through its nitrogen, five- or six-membered heterocyclic sulfide group, malonic ester residue which can be bonded at its α-position, β-ketoester residue which can be bonded at its α-position, or β-diketone residue which can be bonded at its α-position, each optionally being substituted;
X represents halogen, nitro, cyano, R⁸, OR⁸, SR⁸, SO₂R⁸ or NR⁸R⁹;
each of R⁸ and R⁹ represents hydrogen; or each of R⁸ and R⁹ represents C₁-C₆ alkyl, C₂-C₆ alkenyl, C₂-C₆ alkynyl, phenyl or benzyl, each being optionally substituted, and R⁸ and R⁹ of NR⁸R⁹ may be the same or different and may be bonded to each other to form a ring structure;
Y represents O, S, SO or SO₂;
n represents 0, 1 or 2; and
p represents 1 or 2.

2. The benzothiophene derivative according to claim 1, having a structure represented by the formula (I-a): 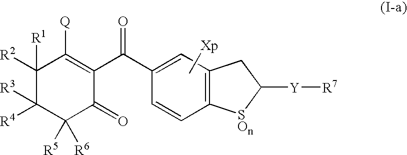
wherein R¹ to R⁷, Q, X, Y, n and p are the same as defined in claim 1.

3. The benzothiophene derivative according to claim 1 or 2, wherein n is 2.

4. The benzothiophene derivative according to any one of claims 1 to 3, wherein R¹ to R⁶ are all hydrogen.

5. The benzothiophene derivative according to any one of claims 1 to 4, wherein X is chlorine, bromine, nitro, cyano or C₁-C₆ alkyl which may be substituted.

6. The benzothiophene derivative according to any one of claims 1 to 5, wherein Y is S, SO or SO₂.

7. The benzothiophene derivative according to any one of claims 1 to 6, wherein Q is OH.

8. A herbicidal composition comprising a benzothiophene derivative as defined in any one of claims 1 to 7 as a herbicidally effective ingredient.

9. The herbicidal composition according to claim 8 for use in paddy rice field.
